(19) United States
(12) Patent Application Publication
(10) Pub. No.: US 2026/0256423 A1
(43) Pub. Date: Sep. 3, 2026

(54) THERAPY FOR THE TREATMENT OF DISORDERS IN THE NERVOUS SYSTEM BY MEANS OF CLASSIFICATION OF DETECTED TREMORS THROUGH MACHINE LEARNING

(71) Applicant: BIOTRONIK SE & Co. KG, Berlin (DE)

(72) Inventors: Sebastian Schulze-Luckow, Berlin (DE); Andrew B. Kibler, Lake Oswego, OR (US); Koeun Lim, Tucson, AZ (US)

(21) Appl. No.: 19/489,943

(22) PCT Filed: Jun. 4, 2024
(86) PCT No.: PCT/EP2024/065266

§ 371 Date: Dec. 4, 2025

Related U.S. Application Data
(60) Provisional application No. 63/471,639, filed on Jun. 7, 2023.

(30) Foreign Application Priority Data
Jul. 4, 2023 (EP) 23183351.8

(51) Int. Cl. A61B 5/00; A61N 1/05; A61N 1/36; G16H 50/20

(52) U.S. Cl. CPC A61B 5/686; A61B 5/7267; A61N 1/0534; A61N 1/36067; G16H 50/20

(57) ABSTRACT

A method includes receiving data for an episode stored by a medical device of a patient. The episode is associated with a period of time. The data includes an electroencephalogram or data derived from neural activity in the brain sensed by the medical device during the period of time. One or more machine learning models are applied to the data and are configured to output a respective likelihood value for each of a plurality of tremor type classifications. The method further includes, based on the application of the one or more machine learning models to the episode data, deriving, for each of the tremor type classifications, class activation data indicating varying likelihoods of the classification over the period of time; and displaying a graph of the varying likelihoods of the tremor type classifications over the period of time.

The present invention relates to a method and a medical system, particularly for classification of detected tremors through machine learning.

A majority of countries have unfavorable age pyramids—this is particularly true for developed industrialized countries. In case a person is older than 65, the risk of a fall is significantly increased. The medical consequences can be more serious than the fall. Falls in older people worldwide pose a major challenge to systems of care. A recognized prerequisite for unfavorable fall prognoses in older people—in this case especially the group over 80 years of age—are so-called intrinsic factors. Neurological disorders, especially dementia and Parkinson's disease, are of importance. Both make physiological locomotion difficult. Parkinson's episodes are accompanied by so-called tremors. Therefore, it is desirable to preventively detect tremors in order to initiate measures to protect elderly patients from falls, e.g., raising awareness, sitting down or holding on.

Tremors are symptoms of diseases like Parkinson's. Particularly, a tremor is an involuntary, often rhythmic, muscle contraction and relaxation involving back and forth movements of one or more body parts. It is one of the most common involuntary movements, and frequently occurs in the hands of a patient, but it can also affect other body parts such as arms, eyes, face, head, vocal folds, trunk, and legs.

Known solutions for diagnosing/treating tremors require visiting a physician for clinical presentation, magnetic resonance therapy of the brain after medical diagnosis, and measurement of muscle activity by means of electrodes in the clinic.

However, all of the above measures typically involve visiting a physician. The typical population of patients is regularly older than 65 years, the repeated visit of the doctor is necessary for the diagnosis of this population. Thus, particularly, only a temporary measurement (time window) is provided, not a permanent monitoring, so that an insufficient diagnosis is a possible outcome. Furthermore, the diagnosis depends on the experience of the physician, wherein such diagnosis usually confirms that a tremor is either present or not, and may describe the quality of the tremor as severe or mild-but will regularly not be given as objectively measured value. Therefore, existing solutions are often partially unnecessary, expensive, time consuming, i.e. they tie up personnel, not comparable to the advance of the state of knowledge, and possibly prone to error as the physician evaluates.

Therefore, based on the above, the problem to be solved by the present invention is to provide a method and a medical system that allow to improve classification of involuntary body movements such as tremors.

This problem is solved by a method having the features of claim 1 as well as by a medical system having the features of claim 6. Preferred embodiments of the respective aspect of the present invention are stated in the corresponding dependent claims and are described below.

According to claim 1, a method is disclosed, comprising:
receiving, by processing circuitry, episode data for an episode stored by a medical device of a patient,
wherein the episode is associated with a period of time, and the episode data comprises an electroencephalogram (EEG) or data derived from neural activity in the brain sensed by the medical device during the period of time;
applying, by the processing circuitry, one or more machine learning models to the episode data, the one or more machine learning models configured to output a respective likelihood value for each of a plurality of tremor type classifications, each of the likelihood values representing a likelihood that the respective tremor type classification occurred at any point during the period of time;
based on the application of the one or more machine learning models to the episode data, deriving, by the processing circuitry and for each of the tremor type classifications, class activation data indicating varying likelihoods of the classification over the period of time; and displaying, by the processing circuitry and to a user, a graph of the varying likelihoods of the tremor type classifications over the period of time.

According to an embodiment of the present invention the machine learning model is a deep convolutional neural network classification model. That type of machine learning model is chosen if the neural activity measurements are in spatiotemporal domain. Given the complexity of the input data, a deep convolutional neural network classification model is a suitable machine learning algorithm for classifying tremor vs non-tremor with spatiotemporal inputs.

According to an embodiment, for training of a convolutional neural network, a training set is required with multi-channel EEG time-series with specific length in time, each labeled with ‘Tremor’ and ‘Not tremor’. For the prediction of tremor, time-series slices, e.g. a sliding window with some overlap, can be fed to the model. The model should be able to generate predictions near-real-time with delay equal to the sum of window length and processing time. General framework for training of a convolutional neural network exists for instance in open source python packages (such as PyTorch).

Alternatively, the machine learning model is a support vector machine. Tremor is characterized in terms of activation location and propagation over time as well as oscillation frequency. When the signal is converted to the frequency domain, certain frequency band may stand out during a specific time range. If such feature is strong enough, a support vector machine is applicable for detecting tremor.

Preferably, to output a respective likelihood value for each of a plurality of tremor type classifications, a neural network classifier is required. For the neural network classifier, each node (neuron) from the output layer outputs the probability of individual classes (i.e. one neuron=one class). For the alternative using a support vector machine as machine learning model, permutation (or resampling) can be performed while training to obtain the probability distribution of the predicted output labels to obtain the inference.

The method according to the present invention thus allows to detect disorders in the nervous system of a patient associated with a rhythmic trembling of a part of the body (tremor). A tremor of the hands is symptomatic of tremors. The invention solves the task of recognizing acute tremors, in particular according to their type or classification, or to predict their onset, in particular according to their nature or classification, by either using data of at least one electroencephalogram (EEG) or data of at least one EEG and wearable device data with a deep learning algorithm. The data can be measured on a patient-wide basis as well as for an individual patient.

According to a preferred embodiment of the method according to the present invention, the episode data further comprises wearable device data obtained by a wearable device worn by the patient. Fir instance, wearable device data includes at least one of movement activity data, electrogram data (e.g. electromyogram), PPG data, heart rate data etc.

Furthermore, according to a preferred embodiment of the method according to the present invention, the wearable device is one of: a smartwatch, a smart necklace, a smart anklet.

Particularly, each of these smart devices is characterized in that it comprises a user interface for displaying information to a user (e.g. patient) and receiving input from the user, and a processor configured to execute a computer program that conducts obtaining the wearable device data, and particularly to preprocess and/or transmit said wearable device data or preprocessed wearable device data to a remote device, particularly to processing circuitry of a computer system, that may comprise at least one server (located e.g. in a medical service center).

According to yet another embodiment of the method according to the present invention, the medical device is a brain stimulator for neural stimulation of the brain of the patient.

Further, in a preferred embodiment of the method, the one or more machine learning models are configured to one of: supervised learning, unsupervised learning, semi-supervised learning, reinforcement learning.

According to yet another aspect of the present invention, a medical system is disclosed, the medical system comprising: a medical device, at least one computer system (e.g. comprising a remote server located in a service center) comprising processing circuitry, wherein the medical system is configured to conduct the method according to the present invention based on a trigger, wherein the episode data is collected over time and across patients.

Particularly, according to an embodiment of the presented method, the episode data is collected (i.e. sensed or provided) by the medical device and received by the processing circuitry. The processing circuitry is further configured to apply one or more machine learning models to the episode data, the one or more machine learning models configured to output a respective likelihood value for each of a plurality of tremor type classifications, each of the likelihood values representing a likelihood that the respective tremor type classification occurred at any point during the period of time, and to derive, based on the application of the one or more machine learning models to the episode data, for each of the tremor type classifications, class activation data indicating varying likelihoods of the respective tremor type classification over the period of time, and to display (e.g. on a suitable display means), to a user, a graph of the varying likelihoods of the tremor type classifications over the period of time.

Furthermore, according to a preferred embodiment of the medical system said trigger is controlled by a time parameter or an event.

Preferably, in an embodiment of the medical system according to the present invention, the event corresponds to receiving new episode data or to receiving a pre-defined number of sets of episode data. In both cases, the new data is used by the medical system to perform calculations for the likelihood that a respective tremor type classification occurred at some period of time.

Furthermore, according to yet another aspect of the present invention, a method is disclosed, the method comprising the steps of:
receiving, by processing circuitry, episode data for an episode stored by a medical device of a patient,
wherein the episode is associated with a period of time, and the episode data comprises accelerometer data (preferably from a 3-axis accelerometer) sensed by the medical device during the period of time;
applying, by the processing circuitry, one or more machine learning models to the episode data, the one or more machine learning models configured to output a respective likelihood value for each of a plurality of posture or activity type classifications, each of the likelihood values representing a likelihood that the respective posture or activity type classification occurred at any point during the period of time;
based on the application of the one or more machine learning models to the episode data, deriving, by the processing circuitry and for each of the posture or activity type classifications, class activation data indicating varying likelihoods of the classification over the period of time; and displaying, by the processing circuitry and to a user, a graph of the varying likelihoods of the posture and activity type classifications over the period of time.

This aspect can be further characterized by the embodiments of the method relating to the episode data comprising an electroencephalogram or data derived from neural activity in the brain sensed by the medical device during the period of time, wherein here, instead of the tremor type classifications said posture and activity type classifications are considered.

Furthermore, according to yet another aspect of the present invention, a method is disclosed, the method comprising the steps of:
receiving, by processing circuitry, episode data for an episode stored by a medical device of a patient,
wherein the episode is associated with a period of time, and the episode data comprises photoplethysmography (PPG) data (e.g. from a PPG sensor) sensed by the medical device during the period of time;
applying, by the processing circuitry, one or more machine learning models to the episode data, the one or more machine learning models configured to output a respective likelihood value for each of a plurality of tremor type classifications, each of the likelihood values representing a likelihood that the respective of tremor type classification occurred at any point during the period of time;
based on the application of the one or more machine learning models to the episode data, deriving, by the processing circuitry and for each of the of tremor type classifications, class activation data indicating varying likelihoods of the classification over the period of time; and displaying, by the processing circuitry and to a user, a graph of the varying likelihoods of the of tremor type classifications over the period of time.

This aspect can be further characterized by the embodiments of the method relating to the episode data comprising an electroencephalogram or data derived from neural activity in the brain sensed by the medical device during the period of time, wherein here, a PPG sensor is used (see above). The PPG sensor provides data to detect oxygenation as well as correlates of blood pressure, and so additional diagnostics are available.

According to a further aspect of the present invention, a medical device system is disclosed, the medical device system comprising:
a medical device configured to:
sense an electroencephalogram (EEG) of a patient (e.g. via a plurality of electrodes) or provide data derived from neural activity in the brain sensed by the medical device during the period of time, and store episode data for an episode, wherein the episode is associated with a period of time, and the episode data comprises the EEG sensed by the medical device during the period of time or comprises said data derived from neural activity in the brain; and
processing circuitry configured to:
receive the episode data,
apply one or more machine learning models to the episode data, the one or more machine learning models configured to output a respective likelihood value for each of a plurality-of tremor type classifications, each of the likelihood values representing a likelihood that the respective tremor type classification occurred at any point during the period of time,
based on the application of the one or more machine learning models to the episode data, derive, for each of the tremor type classifications, class activation data indicating varying likelihoods of the respective tremor type classification over the period of time, and
display, to a user, a graph of the varying likelihoods of the tremor type classifications over the period of time.

According to a preferred embodiment of the medical device system, the processing circuitry-is configured to display the graph in conjunction with the EEG or said data derived from neural activity in the brain.

Furthermore, according to a preferred embodiment of the medical device system, the processing circuitry is configured to indicate on the graph at least one time of higher likelihood for at least one of the tremor type classifications relative to other times on the graph for the at least one tremor type classification.

Furthermore, according to a preferred embodiment of the medical device system the processing circuitry is configured to: indicate, based on the output of the one or more machine learning models, that the at least one tremor type classification occurred at any point during the period of time; and indicate on the graph at least one time of higher likelihood for the at least one tremor type classification in response to indicating that the at least one tremor type classification occurred at any point during the period of time.

Further, according to a preferred embodiment of the medical device system, each of the one or more machine learning models comprises a plurality of layers, and wherein deriving the activation data comprises deriving the activation data from an output of an intermediate layer of the plurality of layers.

Further, according to a preferred embodiment of the medical device system, the intermediate layer comprises a global average pooling layer.

Furthermore, according to yet another preferred embodiment of the medical device system, the one or more machine learning models comprise one or more tremor classification machine learning models, the one or more tremor classification machine learning models configured to output, for each of the plurality of tremor type classifications, a respective set of tremor type likelihood values, each of the tremor type likelihood values of the set representing a likelihood that the respective tremor type classification occurred at a respective time during the period of time, and wherein the processing circuitry is configured to:
apply one or more depolarization detection machine learning models to the episode data, the one or more depolarization detection machine learning models configured to output a set of depolarization likelihood values, each of the depolarization likelihood values of the set representing a likelihood that a depolarization occurred at a respective time during the period of time; and
identify one or more depolarizations during the episode based on the tremor type likelihood values and the depolarization likelihood values.

Furthermore, according to a preferred embodiment of the medical device system, each of the one or more tremor classification machine learning models comprises a plurality of layers, and the processing circuitry is configured to derive the sets of arrhythmia type likelihood values from an output of an intermediate layer of the plurality of layers.

Further, according to a preferred embodiment of the medical device system, to identify the one or more depolarizations based on the tremor type likelihood values and the depolarization likelihood values, the processing circuitry is configured to apply the one or more depolarization detection machine learning models to the episode data and the tremor type likelihood values.

Furthermore, according to a preferred embodiment of the medical device system, to identify the one or more depolarizations based on the tremor type likelihood values and the depolarization likelihood values, the processing circuitry is configured to at least one of:
modify one or more of the depolarization likelihood values based on one or more of the tremor type likelihood values; or modify a depolarization likelihood threshold based on one or more of the tremor type likelihood values.

Furthermore, according to a preferred embodiment of the medical device system, the processing circuitry comprises processing circuitry of a computing device.

Furthermore, according to a preferred embodiment of the medical device system, the medical device is implantable.

According to another aspect of the present invention, a (preferably non-transitory) computer-readable storage medium is disclosed, comprising instructions that, when executed by processing circuitry' of a computing system, cause the computing system to: receive episode data for an episode stored by a medical device of a patient, wherein the episode is associated with a period of time, and the episode data comprises an EEG sensed by the medical device during the period of time or data derived from neural activity in the brain sensed by the medical device during the period of time; apply one or more machine learning models to the episode data, the one or more machine learning models configured to output a respective likelihood value for each of a plurality of tremor type classifications, each of the likelihood values representing a likelihood that the respective tremor type classification occurred at any point during the period of time;
based on the application of the one or more machine learning models to the episode data, derive, for each of the tremor type classifications, class activation data indicating varying likelihoods of the respective tremor type classification over the period of time; and display a graph of the varying likelihoods of the tremor type classifications over the period of time.

In the following, embodiments of the aspects of the present invention as well as further features and advantages of the present invention are described with reference to the Figures, wherein

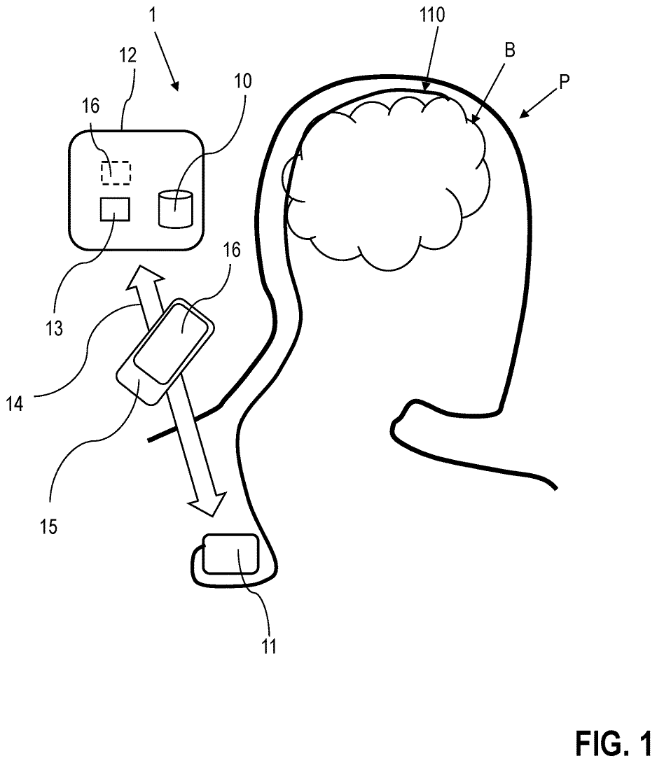

FIG. 1 shows an embodiment of a medical system and method according to the present invention using a deep learning module applied to EEGs to detect tremors of a person; and

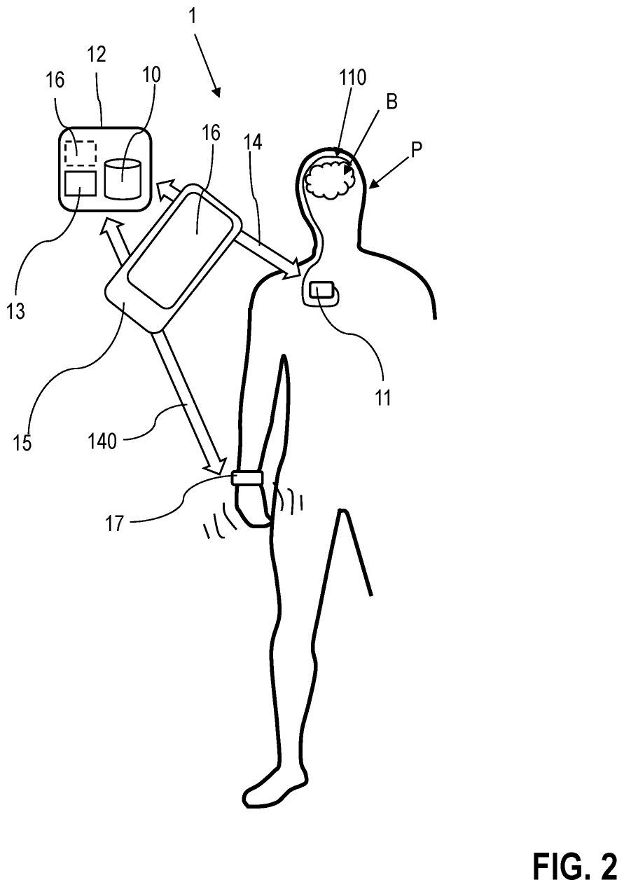

FIG. 2 shows a further embodiment of a medical system and method according to the present invention wherein at least one wearable device worn by a patient can further be used to provide wearable device data as input for the deep learning model.

FIG. 1 is schematic illustration of an embodiment of the method and system according to the present invention. The system 1 and method is adapted to utilize machine learning models to detect and classify tremors of the patient P in accordance with the techniques of the disclosure. The system 1 can comprise a medical device 11 which can be a brain pacemaker for neural stimulation of the brain B of the patient P via suitable electrodes 110. The medical device 11 can be in wireless communication 14 with at least one an external computer system 12 that can be or comprise at least one server in a service center. Said wireless communication 14 can involve at least one further device and/or at least one communication network. Particularly, the medical device 11 can comprise communication circuitry that may include one or more processors, memory, wireless radios, antennae, transmitters, receivers, modulation and demodulation circuitry, filters, amplifiers or the like for radio frequency communication with external devices such as computer system 12. Particularly, the wireless communication 14 can employ an intermediate external device 15 and the computer system 12 can be configured to wirelessly communicate with the medical device 11 and vice versa via the intermediate external device 15 that can be a mobile device. The intermediate external device 15 can be a patient device or a programmer for programming the medical device 11. Particularly, said intermediate external device 15 may provide a user interface comprising a display 16 and allow a user to interact with the medical device 11. Furthermore, the computer system 12 may be configured to allow a user to interact with the medical device 11, or to collect data from the medical device 11, particularly via the wireless communication 14 using said intermediate external device 15.

The at least one computer system 12 comprises processing circuitry 13 for receiving episode data for an episode stored by the medical device 11 of the patient P. The computer system 12 can also comprise a storage device 10 for storing data such as said episode data. The episode data can be transmitted to the computer system 12 via the intermediate external device 15. The episode is associated with a period of time, and the episode data comprises an electroencephalogram (EEG) sensed by the medical device 11 or data derived from neural activity in the brain B sensed by the medical device 11 during the period of time.

Furthermore, the processing circuitry 13 is configured to apply one or more machine learning models to the episode data, the one or more machine learning models configured to output a respective likelihood value for each of a plurality of tremor type classifications, each of the likelihood values representing a likelihood that the respective tremor type classification occurred at any point during the period of time. In this regard, at least one suitable computer program may be executed by the processing circuitry 13 of the computer system 12 to apply said one or more machine learning models to the episode data.

Furthermore, based on the application of the one or more machine learning models to the episode data, the processing circuitry 13 is configured to derive for each of the tremor type classifications, class activation data indicating varying likelihoods of the classification over the period of time; and displaying via a display 16, by the processing circuitry 13, to a user, a graph of the varying likelihoods of the tremor type classifications over the period of time. The display 16 can be a display of said intermediate external device 15. Alternatively, or in addition, the display 16 can also be connected to the computer system 12 or a client thereof as indicated with dashed lines in FIG. 1.

According to a further preferred embodiment shown in FIG. 2, the episode data can further comprise wearable device data obtained by a wearable device 17 worn by the patient P. Particularly, the wearable device 17 is a smartwatch, but can also be one of the other smart devices disclosed herein in this regard. The wearable device data is indicative of movements of the patient P and thus particularly indicative of tremors of the hand in case the wearable device is a smartwatch or anklet. Particularly, this data can in general be gathered for a population of patients or for an individual patient P. The wearable device data portion of the episode data can be transmitted to computer system 12 via a wireless connection 140 that can also be established via intermediate external device 15. Particularly, the intermediate external device 15 may be used to retrieve wearable device data from the wearable device 17 and may transmit the wearable device data to computer system 12. Particularly, the wearable device 17 may also acquire other physiological parameters of the patient P.

Generally, said wireless communication connections 14, 140 may use a network which in turn may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. The network may include one or more networks administered by service providers, and may thus form part of a large-scale public network infrastructure, e.g., the Internet. The network may provide computing devices, such as computer system 12, the intermediate external device 15, the medical device 11 and/or the wearable device 17 access to the Internet, and may provide a communication framework that allows the computing devices to communicate with one another. The network may also be a private network that provides a communication framework that allows computer system 12, and intermediate external device 15, medical device 11 and/or wearable device 17 to communicate with one another but may isolates one or more of computer system 12, intermediate external device 15, medical device 11 and/or wearable device 17 from devices external to the network for security purposes. In some examples, the communications between computer system 12, intermediate external device 15, medical device 11 and/or wearable device 17 are encrypted.

The processing circuitry 13 of computer system 12 can be configured to applying machine learning models to episode data to detect tremors. The processing circuitry 13 may receive episode data for episodes from medical device 11 (FIG. 1) or from medical device 11 and wearable device 17, which may store episode data in response to their detection of a tremor and/or user input. Based on the application of one or more tremor classification machine learning models, the processing circuitry 13 may determine the likelihood that one or more tremors of one or more types occurred during the episode including, in some examples, one or several tremors identified by the medical device 11 that stored the episode data or by the medical device 11 and/or the wearable device 17 that have e.g. stored the respective episode data.

The processing circuitry 13 may also derive and plot (e.g. via display 16) activation data indicating the likelihoods of various tremor type classifications over the time period of the episode, and apply one or more depolarization detection machine learning models to the episode data to identify the occurrence of depolarizations, during the episode. The processing circuitry 13 may display in conjunction with display 16, one or more of the tremor type classifications, tremor type classification likelihood plots, markers indicating the times of identified depolarizations, and the EEG for the episode, which may facilitate a user's review and comprehension of the episode data and the classification(s) by the processing circuitry 13. Although the techniques are described herein as being performed by processing circuitry 13 of computer system 12, the techniques may be performed by processing circuitry of other suitable devices (e.g. intermediate external device 15, medical device 11, wearable device 17).

The machine learning models may include, as examples, neural networks, deep learning models, convolutional neural networks, or other types of predictive analytics systems.

Particularly, computer system 12 receives episode data for episodes stored by medical device 11 and/or wearable device 17. Storage device 10 may store the episode data for the episodes. The episode data may have been collected by the medical device 11 and/or wearable device 17 in response to the medical device 11/wearable device 17 detecting tremors and/or user input directing the storage of episode data. The processing circuitry 13 can be configured to review and annotate the episodes, and generate reports or other presentations of the episodes subsequent to the annotation for review by a clinician or other reviewer. The processing circuitry 13 can utilize further devices of the computer system or other devices to display episode data, tremor type classifications, plots, identified depolarizations, and any other information described herein to users, and to receive any annotations or other input regarding the episode data from the users.

Furthermore, to review and annotate episodes, the processing circuitry 13 of computer system 12 may apply the episode data as inputs to a selected one or more machine learning models. Particularly, the processing circuitry may apply episode data to one or more tremor classification machine learning models and/or one or more depolarization detection models. Machine learning models may include, as examples, neural networks, such as deep neural networks, which may include convolutional neural networks, multi-layer perceptions, and/or echo state networks, as examples.

The tremor classification machine learning models may be configured to output, for each of a plurality of tremor type classifications, values indicative of the likelihood that a tremor of the type occurred at any point during the episode.

Particularly, the processing circuitry 13 can apply configurable thresholds (e.g., 50%, 75%, 90%, 95%, 99%) to the likelihood values to annotate the episode as including one or more tremor types, e.g., based on the likelihood for that classification meeting or exceeding the threshold.

In some examples, tremor classification machine learning models are trained with training data that comprises EEG and particularly also other episode data, such as wearable device data, for a plurality of patients labeled with descriptive metadata. For example, during a training phase, processing circuitry 13 of computer system 12 processes a plurality of EEGs. Typically, the plurality of EEG waveforms is from a plurality of different patients, but may be from a single patient. Each EEG waveform is labeled with one or more episodes of tremor of one or more types.

For example, a training EEG waveform may include a plurality of segments, each segment labeled with a descriptor that specifies an absence of tremor or a presence of a tremor of a particular classification (e.g., based on the signal frequency, amplitude or morphology). In some examples, a clinician labels the presence of tremor in each EEG waveform by hand. In some examples, the presence of tremor in each EEG waveform is labeled according to classification by an EEG feature delineation algorithm.

Particularly, the processing circuitry 13 may be configured to operate to convert the training data into vectors and multi-dimensional arrays upon which the processing circuitry 13 may apply mathematical operations, such as linear algebraic, nonlinear, or alternative computation operations.

Furthermore, processing circuitry 13 can use the training data to teach the one or more tremor classification machine learning models to weigh different features depicted in the EEG data and particularly wearable device data. In some examples, the processing circuitry 13 can use the EEG data to teach the machine learning model to apply different coefficients that represent one or more features in an EEG as having more or less importance with respect to an occurrence of a tremor of a particular classification the same applies to wearable device data being indicative of tremors due to detecting body movement of the patient P (e.g. via an acceleration sensor). By processing numerous such EEG waveforms and particularly wearable device data labeled with episodes of tremor, processing circuitry 13 may build and train one or more tremor classification machine learning models to receive EEG data and particularly also wearable device data from a patient, such as patient P of FIG. 1 or 2, that processing circuitry 13/computer system 12 has not previously analyzed, and process such EEG data/wearable device data to detect the presence or absence of tremor types of different classifications in the patient with a high degree of accuracy. Typically, the greater the amount of EEG data on which the one or more tremor classification machine learning models is trained, the higher the accuracy' of the machine learning models in detecting or classifying tremor in new EEG data or wearable device data.

After the processing circuitry 13/computer system 10 has trained one or more tremor classification machine learning models, the processing circuitry 13 may receive episode data, such as EEG data, for a particular patient, such as patient P. The processing circuitry 13, applies the one or more trained tremor classification machine learning models to the episode data to determine whether one or more tremor types occurred at any point during the episode.

Furthermore, in some examples, processing circuitry 13 may process one or more features of the EEG data and particularly wearable device data instead of, or in addition to, the raw EEG data itself. The one or more features may be obtained via feature delineation performed by medical device 11, and/or wearable device 17, and/or processing circuitry 13. The features may include intervals between features of the EEG or wearable device data, one or more amplitudes, widths or morphological features or other features of the EEG, variability of any of these features. In such example implementations, the processing circuit 13 may train the one or more tremor classification machine learning models via a plurality of training features labeled with episodes of tremor, instead of or in addition to the plurality of EEG waveforms labeled with episodes of tremor as described above.

In further examples, processing circuitry 13 can generate, from the EEG data, an intermediate representation of the EEG data. For example, processing circuitry may apply one or more of signal processing, down sampling, normalization, signal decomposition, wavelet decomposition, filtering, noise reduction, or neural-network based feature representation operations to the EEG data to generate the intermediate representation of the EEG data. Processing circuitry 13 may process such an intermediate representation of the EEG data to detect and classify tremors of various types in patient P. Furthermore, processing circuitry may train the one or more tremor classification machine learning models via a plurality of training intermediate representations labeled with episodes of tremor, instead of the plurality of raw EEG waveforms labeled with episodes of tremor as described above. The use of such intermediate representations of the EEG data may allow for the training and development of lighter-weight, less computationally complex tremor classification machine learning models by processing circuitry 13. Further, the use of such intermediate representations of the EEG data may require less iterations and fewer training data to build an accurate machine learning model, as opposed to the use of raw EEG data to train the machine learning model.

In some examples, based on the application of episode data to the one or more tremor classification machine learning models, processing circuitry 13 may derive, for each of the tremor type classifications, class activation data indicating varying likelihoods of the classification over the period of time of the episode's waveform. For a given tremor type, the amplitude of such likelihood values at different times corresponds to the probability that a tremor is occurring at that time, with higher values corresponding to higher probability.

Furthermore, class activation mapping may make it possible to identify regions of an input time series, e.g., of EEG data and particularly wearable device data, that constitute the reason for the time series being given a particular classification by the one or more tremor classification machine learning models. A class activation map for a given classification may be a univariate time series where each element (e.g., at each timestamp at the sampling frequency of the input time series) may be a weighted sum or other value derived from the outputs of an intermediate layer of a neural network or other machine learning model. The intermediate layer may be a global average pooling layer and/or last layer prior to the output layer neurons for each classification.

Further, processing circuitry 13 may display, e.g., via a display 16 of the computer system 12 of the intermediate external device 15, a graph of the activation data over the time period of the episode. In some examples, the processing circuit may display the class activation data in conjunction with, e.g., on the same screen and at the same time, as the input EEG. While the one or more tremor classification machine learning models may be configured to provide an output that indicates a likelihood of different tremor type classifications occurring during the episode as a whole, the class activation data may allow processing circuitry 13 and/or a user to identify a time during an episode and point during the EEG at which one or more tremors of one or more types likely occurred.

Furthermore, post-processing of episode data stored by the medical device 11 and/or the wearable device 17 may include identifying the occurrences of depolarizations within the EEG data. The identification of depolarizations during post-processing may be different than that by medical device 11 and/or wearable device 17 during detection of the episode and storage of the episode data, providing evidence of a possible misclassification of the episode by medical device 11. Annotation of the EEG data with markers of the occurrence of identified depolarizations may also facilitate review of the episode data by a user and/or processing circuitry 13. Feature delineation techniques to detect depolarizations may include filtering the EEG data, feature extraction (e.g., using a rectified power signal), peak detection, and refractory analysis or other further processing. Such feature delineation may require feature engineering and detection rule development.

Furthermore, processing circuitry 13 can apply one or more tremor classification machine learning models to the episode data, e.g., to the EEG data and particularly wearable device data. The one or more tremor classification machine learning models output a respective likelihood value for each of a plurality of tremor type classifications, each of the likelihood values representing the likelihood that the respective tremor type classification occurred at any point during the period of time. Based on the application of the one or more tremor classification machine learning models to the episode data, processing circuitry 13 can also derive, for each tremor type classification, class activation data indicating varying likelihoods of the classification over the period of time. As described herein, processing circuitry 16 can be configured to derive the activation data from the output of an intermediate layer of machine learning model, such as a final layer before the classification layer and/or a global average pooling layer.

Further, processing circuitry 13 is configured to plot the activation data for the various tremor type classifications over time, and can in particular display a graph of the activation data plots to a user. In some examples, processing circuitry 13 is configured to display the plot in conjunction with the EEG and particularly wearable device data for the episode, which may allow a user to correlate times of relatively high likelihood for a particular tremor type with portions of the EEG and/or wearable device data causing the relatively high likelihood. In some examples, processing circuitry 13 can be configured to indicate, e.g., annotate or highlight, times of higher likelihood for at least one of the tremor type classifications relative to other times of the plot for the at least one tremor type classification. In some examples, when processing circuit 13 indicates based on the output of the one or more tremor detection machine learning models that a tremor of a particular type occurred at some unspecified point during an episode, processing circuitry 13 may further indicate relatively higher likelihood times for that tremor type classification on the plot of activation data so that a user may understand the reasoning for the classification by the one or more tremor detection machine learning models, e.g., by referring to the corresponding portions of the EEG and/or wearable device data.

1. A method, comprising the steps of:
receiving, by processing circuitry (13), episode data for an episode stored by a medical device (11) of a patient (P), wherein the episode is associated with a period of time, and the episode data comprises an electroencephalogram or data derived from neural activity in the brain (B) sensed by the medical device (11) during the period of time;
applying, by the processing circuitry (13), one or more machine learning models to the episode data, the one or more machine learning models configured to output a respective likelihood value for each of a plurality of tremor type classifications, each of the likelihood values representing a likelihood that the respective tremor type classification occurred at any point during the period of time; and
based on the application of the one or more machine learning models to the episode data, deriving, by the processing circuitry (13) and for each of the tremor type classifications, class activation data indicating varying likelihoods of the classification over the period of time; and displaying, by the processing circuitry (13) and to a user, a graph of the varying likelihoods of the tremor type classifications over the period of time.

2. The method according to claim 1, wherein the episode data further comprises wearable device data obtained by a wearable device (17) worn by the patient (P).

3. The method according to claim 2, wherein the wearable device (17) includes one of: a smartwatch, a smart necklace, or a smart anklet.

4. The method according to claim 1, wherein the medical device (11) includes a brain pacemaker for neural stimulation of the brain (B) of the patient (P).

5. The method according to claim 1, wherein the one or more machine learning models are configured to one of: supervised learning, unsupervised learning, semi-supervised learning, or reinforcement learning.

6. A medical system (1), comprising:
a medical device (11); and
at least one computer system (12) comprising processing circuitry (13),
wherein the medical system (1) is configured to conduct the method according to claim 1 based on a trigger, wherein the episode data is collected over time and across patients.

7. The medical system of claim 6, wherein said trigger is controlled by a time parameter or an event.

8. The medical system according to claim 7, wherein the event corresponds to receiving new episode data or to receiving a pre-defined number of sets of episode data.
